(19) United States
(12) Patent Application Publication
(10) Pub. No.: US 2022/0293252 A1
(43) Pub. Date: Sep. 15, 2022

(54) INTELLIGENT SCHEDULING SYSTEM AND METHODS BASED ON PATIENT SPECIFIC DATA AND CLUSTER SIMILARITY

(71) Applicant: CERNER INNOVATION, INC., Kansas City, KS (US)

(72) Inventors: Praveen Bhat Gurpur, Bengaluru (IN); Nandini Muralidharan, Bangalore (IN); Revathi Sankar, Chennai (IN)

(21) Appl. No.: 17/196,766

(22) Filed: Mar. 9, 2021

(51) Int. Cl. G16H 40/20; G16H 10/60; G06Q 10/10; G16H 50/70; H04W 4/029

(52) U.S. Cl. CPC G16H 40/20; G16H 10/60; G06Q 10/1095; G16H 50/70; H04W 4/029

(57) ABSTRACT

Methods, systems, and computer-readable media are disclosed herein for intelligently identifying specific timeslots and identifying specific durations for the timeslots that can be recommended to a person seeking to make an appointment with a clinician. A scheduling module leverages real-time GPS data of the person and clustering techniques to identify the timeslots and predicted durations the person is predicted to utilize in that appointment based at least on a chief complaint and patient cohort data.

### BACKGROUND

Computerized scheduling systems employ appointment templates that have fixed timeslots and static durations. The fixed templates are utilized across different clinicians and various patients. As such, computerized scheduling systems do not provide flexibility in timeslots and duration and therefore they fail to meet the varied scheduling needs of different clinicians and various patients. As a result of the technological limitations of computerized scheduling systems, including inflexible and fixed timeslot/duration templates, “time waste” occurs and patient wait times increase. Furthermore, computerized scheduling systems cannot automatically customize timeslots and durations to fit the medical needs of each unique patient.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims as supported by the Specification, including the Detailed Description.

In brief and at a high level, this disclosure describes, among other things, methods, systems, and computer-readable media for leveraging near real-time geographic location information in tandem with appointment data and medical data of patient cohorts to calculate future appointment start times and predicted durations that are tailored to the clinical needs of an individual patient. The aspects herein can intelligently identify available timeslots for multiple patients who wish to have an appointment with a clinician and can predict the appointment duration that each individual patient would find optimal based on medical data. For example, the available timeslots and the predicted appointment duration for each individual patient can also be determined by estimating the time it will take each patient to travel to a clinical site for the appointment, based on GPS data of that patient. Also, the available timeslots and the predicted appointment duration for each individual patient can also be determined based on patient-specific medical data, such a chief complaint, one or more prior appointments, and/or patient population data clusters matched to the patient. These aspects create flexible scheduling timeslot/duration options of patients and clinicians, to reduce time waste and create “zero wait times” for patients. Furthermore, these aspects automatically customize timeslots and durations to fit the medical needs of each unique patient.

In one aspect, a method is provided that leverages near real-time location information and historical appointment data of patient cohorts to calculate future appointment start times and predicted durations. In aspects, T1 is determined for a first patient, wherein T1 is associated with a geographic location of the first patient. T2 is determined for the first patient, wherein T2 is associated with a user input travel start time, in aspects. T3 is determined, wherein T3 is based on one or more prior appointment durations for a plurality of patients that have the same chief complaint as the first patient, in various aspects. In aspects, T4 is determined for the first patient, wherein T4 is associated with a prior appointment duration of the first patient. When T3 is determined to be greater than T4, in some aspects, one or more future timeslots are identified and communicated to a device associated with the first patient based on T3. When T3 is determined to be less than or equal to T4, one or more future timeslots are identified and communicated to a device associated with the first patient based on T4. A notification can be received from a device associated with the first patient, in aspects, as selecting one of the one or more future timeslots that were communicated to the device.

In another aspect, one more non-transitory computer-readable media are provided that have computer-executable instructions embodied thereon that when executed by a processor perform a method. In such aspects, an identifier of a first patient, a chief complaint of the first patient, and a geographic location of a device associated with the first patient are received. In aspects, confirmation of the geographic location of the device associated with the first patient is received. In various aspects, T1 and T2 are determined for a first patient, wherein T1 is associated with a geographic location of the first patient and T2 is associated with a user input travel start time. In aspects, T3 is determined based on one or more prior appointment durations for a plurality of patients having the same chief complaint as the first patient. T4 is determined for the first patient, in some aspects, wherein T4 is associated with a prior appointment duration of the first patient. When T3 is determined to be greater than T4, one or more future timeslots are identified and communicated to a device associated with the first patient based on T3, in some aspects. When T3 is determined to be less than or equal to T4, in aspects, one or more future timeslots identified and communicated to a device associated with the first patient based on T4. A notification from a device associated with the first patient is received, wherein the notification includes a selection of one of the one or more future timeslots.

In yet another aspect, a system is provided that leverages near real-time location information and historical appointment data of patient cohorts to calculate future appointment start times and predicted durations. In aspects, the system includes a processor, a memory, a patient intake module, and a scheduling module. The patient intake module receives an identifier of a first patient, a chief complaint of the first patient, and a geographic location of a device associated with the first patient, in various aspects. The patient intake module receives, in some aspects, a confirmation of the geographic location of the device associated with the first patient. In various aspects, the scheduling module determines T1 and T2 for a first patient, wherein T1 is associated with a geographic location of the first patient and wherein T2 is associated with a user input travel start time. The scheduling module also determines T3 based on one or more prior appointment durations for a plurality of patients having the same chief complaint as the first patient. The scheduling module determines T4 for the first patient, wherein T4 is associated with a prior appointment duration of the first patient. When scheduling module determines that T3 is greater than T4, one or more future timeslots are identified and communicated to a device associated with the first patient based on T3, in such an aspect. When the scheduling module determines that T3 is less than or equal to T4, the scheduling module identifies and communicates one or more future timeslots to a device associated with the first patient based on T4. A notification from a device associated with the first patient is received by the scheduling module, wherein the notification includes a selection of one of the one or more future timeslots.

### BRIEF DESCRIPTION OF THE DRAWINGS

Aspects are described in detail below with reference to the attached drawings figures, wherein:

FIG. 1 is a block diagram of a system in accordance with one or more aspects of the present invention;

FIGS. 2-6 depict examples of graphical user interfaces (GUI) in accordance with one or more aspects of the present invention;

FIGS. 7A-D depicts a scheduling table that illustrates examples of scheduling multiple patients in accordance with one or more aspects of the present invention; and

FIG. 8 is a flow diagram showing a method in accordance with one or more aspects of the present invention;

FIG. 9 is a flow diagram showing another method in accordance with one or more aspects of the present invention;

FIG. 10 depicts an example computing environment suitable for implementing aspects of the present invention.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

Aspects hereinafter provide a system, methods, and computer-readable media that leverage near real-time location information of a particular patient in tandem with historical appointment data and medical data of clustered patient cohorts in order to identify and recommend future appointment start times and patient-customized appointment durations for booking. These aspects utilize clustering of patient population data to determine a predicted appointment duration to be offered for one or more future timeslots that are available for scheduling a particular patient. Additionally, the predicted appointment duration of the future appointment(s) that is/are available can be determined based on historical clinician-specific data and/or historical clinical site-specific data (e.g., specific to hospital A, neurology clinic B, general clinic C, drive-up laboratory/collection site D, or medical imaging site F) that record the start time, stop time, and total duration of a plurality of prior appointments, for example, as associated with one or more clusters of the patient population data that is identified as a best fit, a closest match, and/or is the most similar to a particular patient for which the appointment is being scheduled. In aspects, one or more future appointment start times and predicted appointment durations are determined in near real-time as an individual patient, for example, utilizes an application to request an appointment with a physician or other clinician. As such, the aspects described hereinafter create technological improvements over other computerized scheduling systems because these aspects perform patient cohort clustering (involving health information) in tandem with a patient's real-time location data (such as GPS) to provide recommended scheduling options and predicted appointment durations that are specific and customized for an individual patient. At a real-world level, the computerized and intelligent scheduling produced via the system, method, and computer-readable media described hereinafter can facilitate reductions in wait times for patients, including the ability to achieve “zero wait” durations.

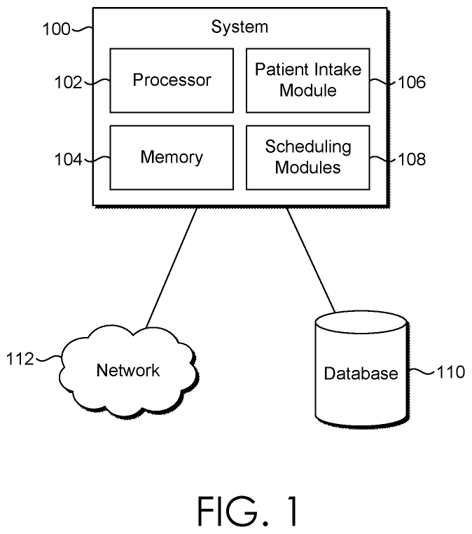

Beginning with FIG. 1, it depicts a block diagram of a system 100 in accordance with one or more aspects of the present invention. The system 100 leverages near real-time location information of a particular patient and historical appointment data of patient cohorts in order to determine and recommend future appointment start times, as well as to predict an appointment duration for the recommended future appointments. The system 100 includes a processor 102, a memory 104, a patient intake module 106, and a scheduling module 108. The system 100 may include or communicate with a database 110 and network 112, as shown in the example of FIG. 1.

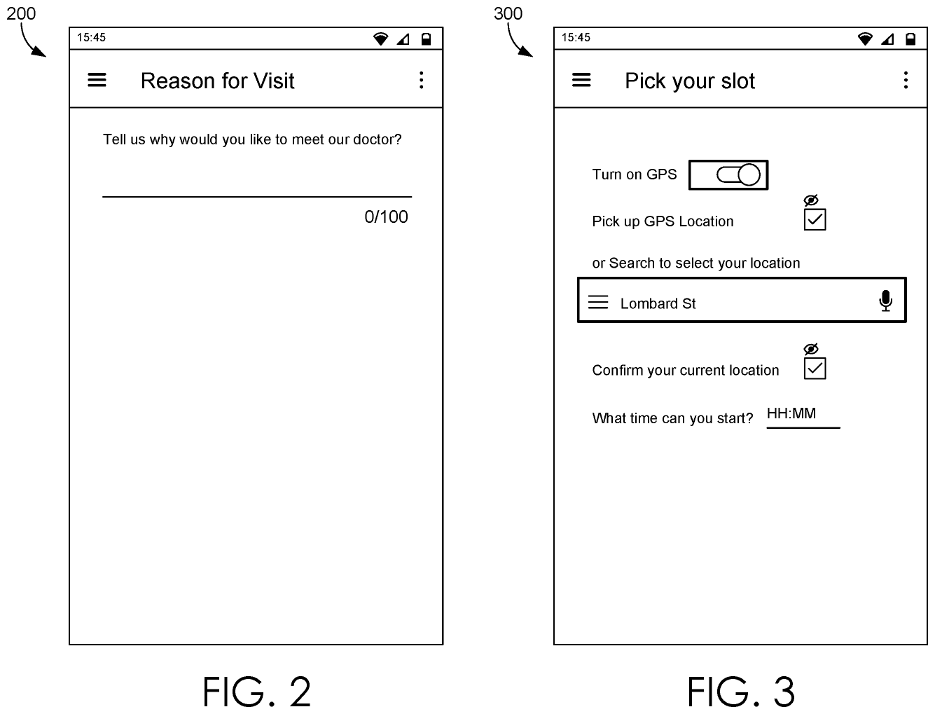

The patient intake module 106 can request and/or receive one or more identifiers of a first patient. For example, the patient intake module 106 may receive a login and password combination that correspond to the first patient. In another example, the patient intake module 106 may receive a full name, a date of birth, a social security number, a medical record number, an address, an electronic token, a machine-readable code, or any combination thereof that can be used as a unique identifier of the first patient. Additionally, the patient intake module 106 can request and/or receive one or more chief complaints of the first patient, for example, wherein the chief complaint is the first patient's reason or rationale for seeking to book an appointment with a clinician. FIG. 2 provides an example GUI 200 that prompts a user, such as a first patient, to manually input one or more chief complaints. Alternatively, one or more chief complaints may be automatically presented as options for selection by the user based on, for example, the first patient's history and/or other patients. The first patient's identifier and/or one or more chief complaints can be provided by the user, such as a first patient or guardian, for example, by manually entering the information into a GUI or other user interface, passively through interrogation of a device that is associated with the first patient, by entering a unique alphanumeric code, or a combination thereof.

In various aspects, the patient intake module 106 requests and/or receives a geographic location representing the physical location of a first patient and/or of a device that is associated with the first patient. The geographic location represents the physical location of a first patient. The geographical location may be manually entered by a user, for example, as manually input via a GUI or other user interface (e.g., text, speech), may be provided actively or passively through an electronic interrogation conducted by the patient intake module 106 of the device that is associated with the first patient, or any combination thereof. The geographic location of the first patient and/or of the device that is associated with the first patient may include Global Positioning Satellite (GPS) coordinates, in one example. In another example, or in addition to receipt of GPS coordinates, the geographic location may comprise a street address (e.g., 123 ABC Street, Central City, and/or State), a postal code (e.g., zip code), a proximity to a particular cell tower, proximity to a particular wireless network, an IP address, or any combination thereof. Generally, the geographic location of the patient is a location from which the first patient will be traveling from to reach a clinical site for the appointment that has not yet been booked. For example, FIG. 3 provides an example GUI 300 that includes multiple options for obtaining the geographic location of the first patient and/or of the device of the first patient (e.g., a touchscreen on/off switch can be toggled to turn on and turn off the optional sharing of GPS coordinates of the device with the patient intake module 106; an input text box that can be used to enter text of a street address, city, state, region, or zip code). In some aspects, the patient intake module 106 may request or prompt the user, such as the first patient, to provide and send a confirmation of the geographic location. Subsequently, the patient intake module 106 can receive a confirmation of the geographic location, though receipt of a confirmation may be optional for the system 100 to continue. In various aspects, the patient intake module 106 communicates or “hands off” the first patient's information, such as one or more first patient identifiers, one or more chief complaints of the first patient, and/or the geographic location associated with the first patient, to the scheduling module 108.

The scheduling module 108 is configured to determine “T1” or “time one” for a first patient. T1 is associated with the geographic location of the first patient and/or the device associated with the first patient. In some aspects, T1 can be determined by aggregating geographic location information (e.g., IP address, GPS coordinates, device identifier), generating a request for an estimated travel time, communicating the request to an external service, and receiving a response from the external service that includes an estimated travel time. For example, a request may be communicated by the scheduling module 108 to an external application or service. The request can include the geographic location of the first patient and a geographic location of a clinical site (e.g., hospital A, neurology clinic B, general clinic C, drive-up laboratory/collection site D, medical imaging site F), which the external application or service can use to estimate a travel time from one location to the other based on a travel direction, one or more routes, one or more possible travel modes, weather conditions, traffic conditions, etc. Subsequently, an estimated travel time may be received by the scheduling module 108 from the external application or service.

The estimated travel time can be used to define T1, in various aspects. The estimated travel time refers to a computer-estimated duration of time that is expected to lapse/be incurred if/when the first patient travels the distance measured from the first patient's geographic location to the clinical sites' geographic location, based on one or more modes of transportation, one or more predefined transportation, and historical data. Modes of transportation may include, for example, modes such as an automobile, a bus, a subway, a train, or walking. Predefined transportation routes may include roads, sidewalks, bus routes, train routes, and the like, for example. The combination of a particular mode of transportation and a particular predefined transportation route that is usable for the first patient to travel to the clinical site from a specified geographic location is thus used to determine or obtain an estimated travel time.

The scheduling module 108 also determines “T2” or “time two” for the first patient. T2 is associated with an input travel start time. The first patient, guardian, or other user booking the appointment on behalf of the first patient may input a travel start time by specifying a time, e.g., 00 hours, 00 minutes. In some aspects, the scheduling module determines T2 by calculating an earliest travel start time that the first patient can begin traveling from the specified geographic location to the clinical site. In some aspects, the scheduling module 108 may request or provide a prompt to enter a travel start time (e.g., date, hour, and minute) from the first patient, guardian, or other user booking the appointment on behalf of the first patient. Subsequently, the confirmation of the travel start time is received from the device that is associated with the first patient, guardian, or other user booking the appointment on behalf of the first patient. Turning again to FIG. 3, it provides the example GUI 300 for requesting a travel start time in the format of hours and minutes of the current date or future date (e.g., “What time can you start?”).

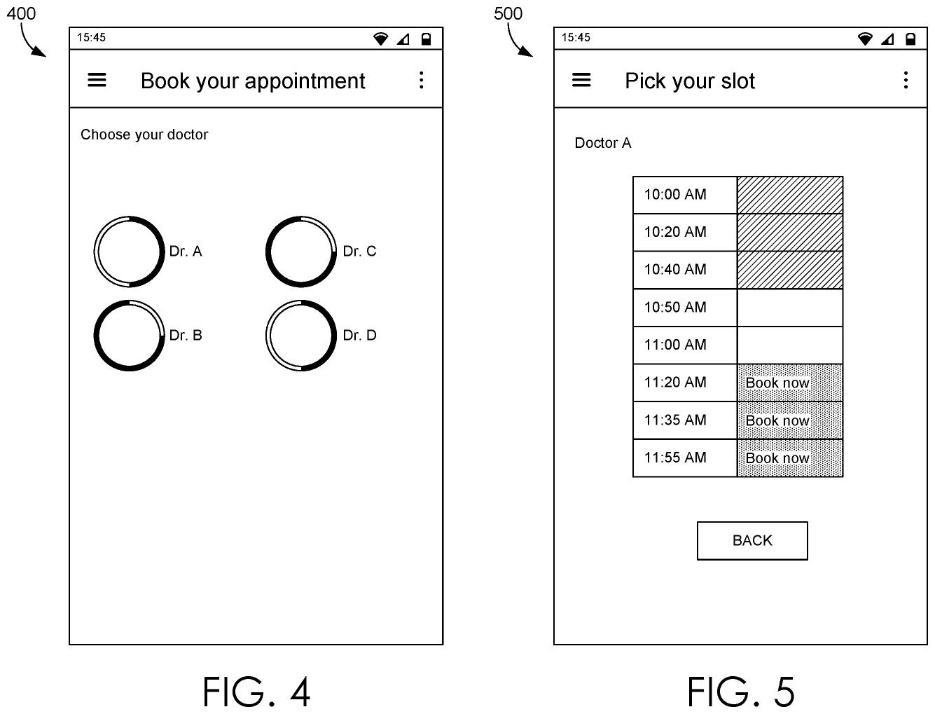

Additionally, the scheduling module 108 can request and/or receive a preferred clinician identifier, for example, that identifies a particular clinician that the first patient would like to schedule the one or more future appointments based on the one or more chief complaints. For example, FIG. 4 depicts an example GUI 400 for requesting a preferred clinician identifier, by way of presenting available clinicians (e.g., via identifier such as a name) as well as visual indicators of appointment availability (e.g., open timeslots on a particular date for which the appointment is sought). Based on the preferred clinician indicator that is selected, received, or identifier by selection, the scheduling module 108 can identify one or more specific geographic locations of clinical sites that is/are associated with one or more of the preferred clinician identifier, and where the sought appointment is to be scheduled. Alternatively, a preferred clinician indicator may be received via the patient intake module 106 which can identify the specific geographic locations that is/are associated with one or more of the preferred clinician identifier. Then, the scheduling module 108 may use the specific geographic locations that is/are associated with one or more of the preferred clinician identifier, provided by the patient intake module 106, relative to the specific geographic location of the first patient and/or the device associated with the first patient in order to determine T1 (i.e., estimated travel time). The scheduling module 108 may concurrently or sequentially determine T1 and T2 based on any order of input from a first patient or guardian. In some aspects, the preferred clinician identifier can be utilized by the patient intake module 106 and/or used by the scheduling module 108 to identify one or more clinical sites that are stored in electronic association with the preferred clinician identifier. The geographic location of the one or more clinical site(s) can be provided to the external service, as previously described, in order to return an estimated travel time relative to the geographic location of the first patient. As such, it should be noted that the sequence of the GUIs shown in FIGS. 2-6 are not fixed and other combinations, orders, or a single continuous GUI are all contemplated to be within the scope of the present invention such that the GUIs are merely examples and should not be construed as limiting aspects of the invention.

Continuing, the scheduling module 108 can determine “T3” or “time three.” T3 is determined based on one or more prior appointment durations for a plurality of patients that have the same chief complaint as the first patient, in aspects. Generally, T3 is determined using patient population data of a plurality of patients that encodes, for example, one or more unique patient identifiers, one or more corresponding demographics, one or more corresponding chief complaints, corresponding appointment(s) information (e.g., a total quantity of appointments, duration of each appointment, one or more chief complaints associated with each particular appointment, a specific clinician associated with each appointment), or any combination thereof. In one example, T3 can be determined, via the scheduling module 108, by referencing data for a population of patients and identifying the portion of the patients that reported, at least, the same chief complaint as the first patient. The patient data may be stored in the database 110 and queried by the scheduling module 108, for example, via the network 112. The patient population data may include, for each patient and/or for each appointment of each patient, a duration for an initial appointment, duration(s) of one or more follow-up appointments, travel times associated with each patient for each of the appointment(s), and combinations thereof, for example. The patient population data may also store demographic information for each of the plurality of patients (e.g., 100,000+ patients). Using the patient population data, the scheduling module 108 can determine T3 by calculating an average time duration spent by one or more clinicians (e.g., different clinicians in different specialties, different clinicians within one specialty, the same clinician as specified from the first patient) for the plurality of patients having the same one or more chief complaints, by calculating an average time duration follow-up consultation time taken for the plurality of patients having the same chief complaint (e.g., for the same or different clinician and/or same clinical site), by calculating an average time duration per patient to reach the clinical site for the plurality of patients having the same chief complaint, or any combination thereof.

The scheduling module 108 determines “T4” or “time four” for the first patient. T4 is associated with a prior appointment duration of the first patient, when present in some aspects. Generally, T4 is determined using patient-specific information (when present) and cluster-specific information, as further described hereinafter. When the first patient is not a new patient for the clinician and/or clinical site, the database 110 stores information regarding durations of one or more appointments of the first patient, clinical sites visited by the first patient, one or more previous recorded chief complaints of the first patient, demographics of the first patient, one or more clinicians that have seen or treated the first patient, location information of the first patient, diagnoses, problems, or any combination thereof, in some aspects. When the first patient is a new patient, information such as the first patient's identifier, one or more chief complaints, demographics, or any combination thereof may be obtained through the patient intake module 106 or via another pre-registration process, for example.

In determining T4 for the first patient, the scheduling module 108 performs computerized clustering of the patient population data without human intervention. For example, the scheduling module 108 uses the patient population data as input and generates clusters of patient cohorts using, for example, one or more of vector quantization techniques (e.g., k-means clustering) and one or more distance measurements between vectors/clusters (e.g., Levenshtein distance). In one example, the patient population data is organized by the scheduling module 108 into clusters based on a plurality of age ranges (e.g., age of 0-18, 18-29, 30-39, 40-49, 50-65, 65+ years, etc.). Additionally or alternatively, the patient population data can be clustered using multiple parameters. For example, the patients may be grouped into clusters using age range(s), gender(s), one or more demographics, chief complaint(s), zip code(s), location(s), association with one or more clinicians (e.g., a clinician a patient has visited), individual medical history, family medical history, problems, diagnoses, modes of travel, appointment history, or any combination thereof. These clusters are not static, however. The patient population data stored in the database 110 can be automatically and periodically updated using data mined from an electronic hospital records (EHR) system. For example, the patient population data can be re-analyzed to generate new clusters, for example, every 15 days, once a week, monthly, annually, biweekly or other timeframe, by retrieved up-to-date EHRs. With each update that infuses the system 100 with new data, the patient population data is analyzed and regrouped into clusters, in various aspects.

In order to leverage the clustered patient population data, information that is specific to the first patient can be obtained through an EHR system or other means. For example, specific information or “parameters” can be mined from an intake form, a mobile application, and/or a patient registration form, or the like for the first patient. This information about the first patient can be used to identify which one or more of the plurality of clusters is a best match, closest fit, and/or has a greatest similarity of its patient cohort relative to the parameters of the first patent. In aspects, one cluster is identified as including a patient cohort having the greatest similarity to the first patient based on the information available for the first patient. For example, a first patient is female, Caucasian, aged 43 years with a chief complaint of “high fever” can be matched to one cluster in the plurality of clusters generated from the patient population data, wherein that one cluster corresponds to a plurality of patients that are female, Caucasian, have an age range of 40-45 years, and have presented at least once with a chief complaint of “high fever,” as this patient cohort is the most similar to the first patient relative to the other clusters.

Having identified a cluster for the first patient, the scheduling module 108 examines one or more prior appointments of the patients that belong to the cluster selected as having the greatest similarity to the first patient for the purpose of determining T4. When available, the scheduling module 108 examines one or more prior appointments of the first patient, in aspects. The scheduling module 108 can identify the duration of each of the one or more prior appointments of the first patient and/or durations of each of the one or more prior appointments for the patients within the cluster selected as having the greatest similarity to the first patient. The scheduling module 108 can also identify, for each one or more appointments of the patients in the population and/or the first patient (when present), one or more chief complaints, one or more geographic locations of a clinical site (e.g., hospital A, neurology clinic B, general clinic C, a drive-up laboratory/collection site D, medical imaging site F), one or more clinicians, one or more clinical specialties, or any combination thereof that are associated with each of the particular appointment(s).

Using this information from the cluster and/or from the first patient EHRs, the scheduling module 108 can calculate the average duration of time incurred for appointments based on the duration of all the appointments of the first patient (when present) and/or based on the duration of time incurred for appointments of the patients within the cluster selected as having the greatest similarity to the first patient, in various aspects. In further aspects, the scheduling module 108 can calculate the average duration of time of an appointment based on the duration of all the appointments of the first patient (when present) with a specific portion of appointments of the patients in the cluster selected as having the greatest similarity to the first patient, wherein the specific portion of appointments are associated with the same clinical site, clinician, clinical specialty, chief complaint, or any combination thereof, as related to the appointment to be scheduled for the first patient. As such, appointments of the first patient alone may be considered, appointments of other patients from the clusters may along be considered, and both can be considered together when determining T4. Further, the scheduling module 108 may, when determining T4, only consider appointments of the first patient and/or cluster identified that are associated with the same chief complaint of the first patient and/or one or more related or similar chief complaints. Alternatively, the scheduling module 108 may, when determining T4, only consider appointments of the first patient and/or cluster identified that are associated with any chief complaint (e.g., T4 determined independent of the chief complaint at issue).

In another example, the scheduling module 108 may calculate the average duration of a plurality of appointments for patients, wherein the appointments are associated with the same clinician that the first patient is attempting to schedule with, and wherein the appointments are also associated with the same chief complaint specified by the first patient. Additionally or alternatively, the scheduling module 108 may calculate the average duration based on one or more prior appointments of the first patient, where the one or more prior appointments are associated with the same clinician. As such, the average duration calculated can be specific to the first patient's appointment history (when present) and/or specific to a particular situation of the first patient (e.g., clinician specific, specific to the chief complaint, specific to a repeatedly reported chief complaint of the first patient's due to a chronic illness in other appointments). The average duration may be used by the scheduling module 108 as the value for T4, in various embodiments.

Generally, for each appointment that is being used to determine the average appointment duration, the actual duration of each appointment can be calculated by the scheduling module 108 using electronically recorded timestamps that are captured and defined by user interactions within one or more clinical workflows. For example, a first metric may determine the duration of an appointment based on timestamps of a clinician opening a chart and closing a chart (e.g., start and stop times of a nurse or other practitioner covering an intake portion of an appointment), timestamps of a clinician typing/entering a problem or diagnosis into an electronic record and closing that instance of the electronic record (e.g., start and stop times of a physician's portion of an appointment), or a combination thereof (e.g., summing these durations of the portions to determine a total appointment duration).

In accordance with the system 100, the scheduling module 108 can compare T3 and T4 of the first patient to determine a predicted appointment duration that is customized and tailored to the first patient. When T3 is determined to be greater than T4 (i.e., T3>T4), the scheduling module 108 identifies and communicates one or more future timeslots to a device associated with the first patient based on T3. The one or more future timeslots include one or more of the date, a start time, a stop time, and/or a predicted duration (i.e., a future start time spanning a length of time to a future stop time). Specifically, the one or more future timeslots are identified as having a future start time that is greater than or equal to T1+T2, and the greater value of T3 is used to determine the predicted duration of the appointment that is customized for the first patient, in this example. Further, T3 can be used to determine the predicted end time of the appointment, which can be used by the scheduling module 108 when providing available timeslots to other users, as discussed hereinafter. One or more available appointments/timeslots within the schedule of a clinician that have a start time at or after T1+T2, and which accommodate the duration of T3, are retrieved by the scheduling module 108 as options for a first patient, guardian, or other user to select in order to book the desired appointment. These available appointments/timeslots within the schedule of a clinician are presented to the first patient or guardian for selection.

When T3 is determined to be less than or equal to T4 (i.e., T3<T4), the scheduling module 108 identifies and communicates one or more future timeslots to a device associated with the first patient based on T4. The one or more future timeslots include one or more of a date, a start time, an end time, and/or a predicted duration (i.e., a future start time spanning a length of time to a future stop time). Specifically, the one or more future timeslots are identified as having a future start time that is greater than or equal to T1+T2, and the greater value of T4 is used to determine the predicted duration of the appointment, in this example. Further, T4 can be used to determine the predicted end time of the appointment for the one or more future timeslots, which can be used by the scheduling module 108 when providing available timeslots to others, as discussed hereinafter. One or more available appointments/timeslots within the schedule of a clinician that have a start time at or after T1+T2 and which allow the duration of T4 are retrieved by the scheduling module 108 as options for selection to schedule the desired appointment. These available appointments/timeslots within the clinician's schedule are presented to the first patient, guardian, or other user on behalf of the first patient for selection. For example, FIG. 5 provides an example GUI 500 illustrating one or more recommended timeslots within the schedule of a particular clinician that have a start time at or after T1+T2, having a duration that can accommodate the greater of T3 or T4, wherein the available timeslots are shown with the designation “Book Now” for selection and booking the desired timeslot for the appointment for the chief complaint.

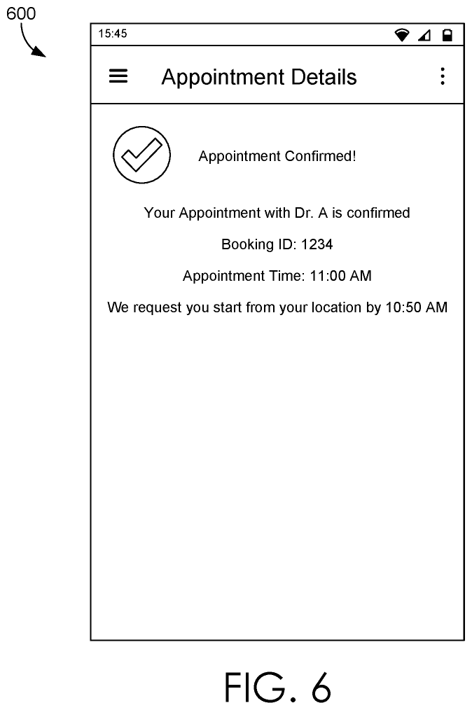

Subsequently, the scheduling module 108 receives a notification from a device associated with the first patient, wherein the notification includes a selection of at least one of the one or more future timeslots. The scheduling module 108 electronically books an appointment for the first patient at one of the selected future timeslot(s) (e.g., selected via GUI 500 in FIG. 5) with the desired clinician (e.g., selected via GUI 400 in FIG. 4), and further associates the appointment that is booked for that timeslot with the first patient identifier, the one or more chief complaints specified (e.g., entered via the GUI 200 of FIG. 2), and optionally, additional information obtained from one or more prior appointments of the first patient (when available). The scheduling module 108 can provide or communicate a confirmation of the successful appointment booking along with appointment details to the first patient, guardian, or other user. For example, FIG. 6 depicts an example GUI 600 that displays the successful appointment booking along with appointment details.

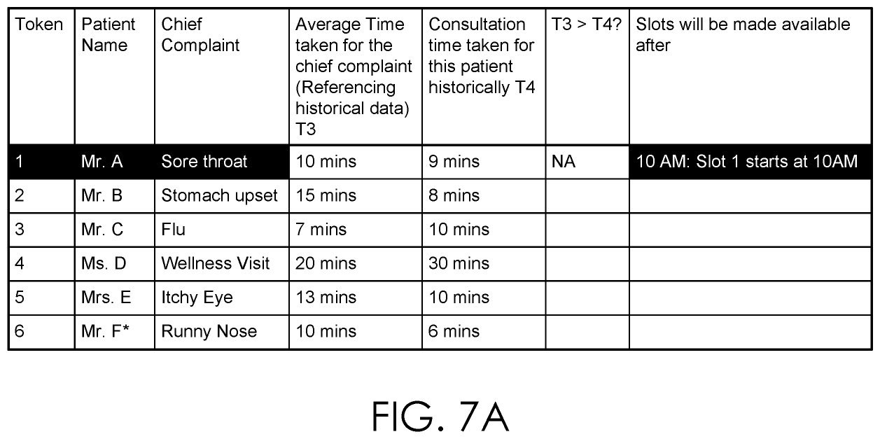

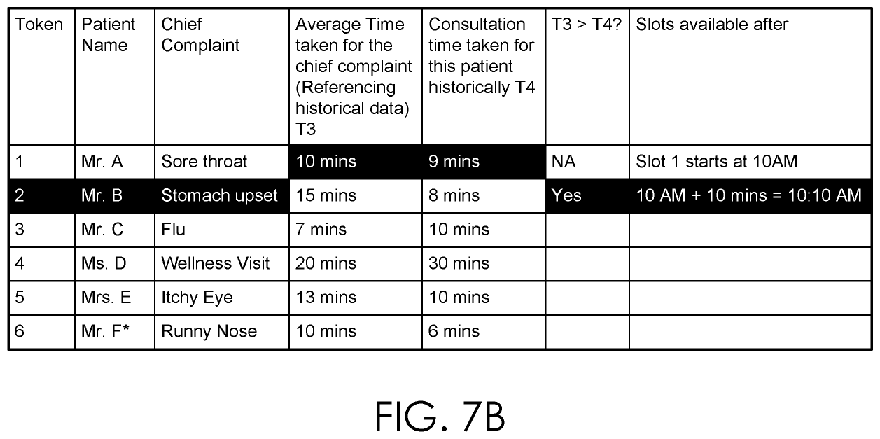

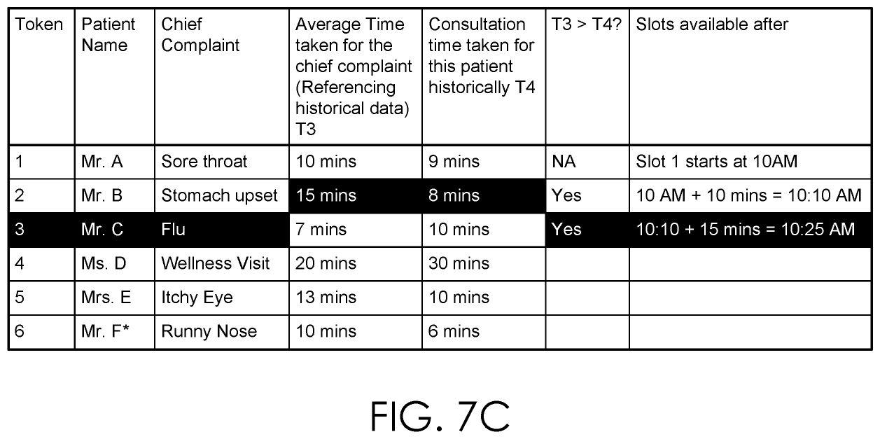

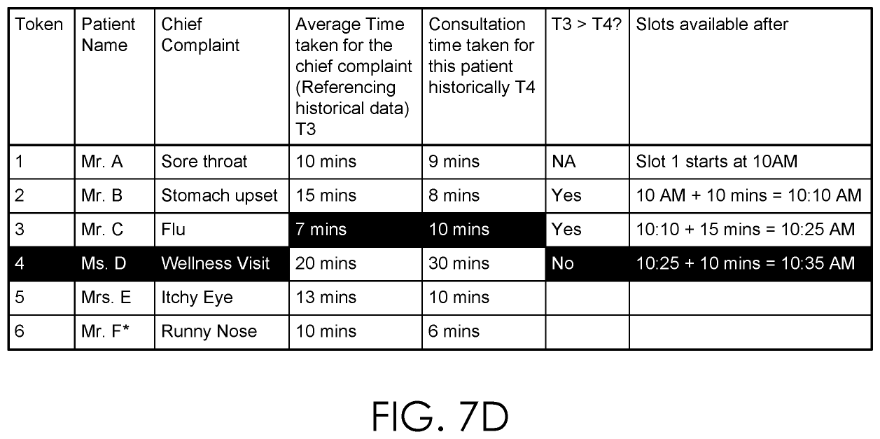

The system 100 shown in FIG. 1 can concurrently be accessed and utilized by any number of people/entities (e.g., thousands or more patients, guardians, user) at any time, as the people/entities utilize the system 100 to book appointments with various clinicians, at various clinical sites, for any number of chief complaints. As such, available timeslots/booked appointments are being updated by the scheduling module 108, in real-time, as multiple people/entities book appointments and/or cancel appointments with various clinicians. In particular, the scheduling module 108 can “stack” appointments to create practical efficiencies, and can shift the available timeslots that it is providing and/or subsequently provides to other people/entities. For example, FIGS. 7A-D depict a scheduling table that illustrates an example of appointment stacking for multiple available timeslots/booked appointments of many patients scheduling with one clinician on a particular date. In FIGS. 7A-D, several patients are using the system 100 to find available timeslots and to schedule an appointment with one clinician on a particular date. The scheduling module 108 has visibility of all the persons attempting to schedule an appointment in real-time, and all the existing/currently scheduling appointments for each of the clinicians at a clinical site. The scheduling module 108 leverages this omnipotent visibility in real-time when identifying and selecting the available timeslots to provide to a person for selection and booking, based on clustering.

For example, in FIG. 7A, a first patient “Mr. A” having reported a chief complaint of a “sore throat” is seeking an appointment with a clinician, this information having been obtained through the patient intake module 106. Based on the geographic location of Mr. A and the geographic location of the clinical site of the clinician with whom an appointment is to scheduled, the scheduling module 108 can identify T1 (i.e., a duration of time that it is predicted it will require or take for Mr. A to travel to the clinical site of that clinician from his reported geographic location based on GPS coordinates) and can identify T2 (i.e., a date and time when Mr. A can begin traveling to the clinical site from his specified geographic location). Additionally, the scheduling module 108 determined T3 and T4 for Mr. A. In this example, T3 is determined to be 10 minutes for Mr. A and corresponds to the average duration of other patients' prior appointments that had the same chief complaint of a sore throat and/or other similarities. In this example, T4 is determined to be 9 minutes for Mr. A and corresponds to the average duration of prior appointments of Mr. A with the clinician with whom an appointment is to be scheduled. In FIG. 7A, a first available timeslot for the date of the appointment to be scheduled has a start time of 10:00 am. This first available timeslot is automatically provided to Mr. A independent of whether T3 or T4 is greater, so long as T1+T2 of Mr. A (i.e., estimated arrival time of Mr. A to the clinical site) provides an earlier or equal start time than 10:00 am. In this example, one or more available timeslots (including the first available timeslot) can be provided to Mr. A for selected and booking. In FIG. 7A, Mr. A selects the first timeslot to book the appointment and the scheduling module 108 books the appointment for Mr. A. This prevents anyone else from booking that timeslot, and the scheduling module 108 will not recommend to anyone else and will prevent anyone else from booking the 10:00 am timeslot of the clinician on the date.

Then in FIG. 7B, the scheduling module 108 continues to stack and shift the one or more available/future timeslots being provided to other patients seeking to schedule an appointment. Because Mr. A has booked the timeslot of 10:00 am and because Mr. A's T3 is 10 minutes (where T3>T4), the scheduling module 108 determines that the following patient Mr. B can be provided with one or more available timeslots that have a start time at or after 10:10 am (i.e., a start time of the first timeslot+T3 as the predicted appointment duration of Mr. A). The scheduling module 108 has determined that Mr. A is predicted to have the scheduled 10:00 am appointment stop time of 10:10 am, and has specifically chosen the greater of T3 and T4 in making the determination. Based on this, the scheduling module 108 determines and recommends one or more available timeslots to Mr. B that have a start time after the predicted appointment stop time of Mr. A, 10:10 am. Accordingly, the greater of Mr. A's T3 or T4 is used to define possible starts times of later timeslots for subsequent patient such as Mr. B. In this same manner, the scheduling module 108 will utilized the greater of Mr. B's T3 or T4 to determine a predicted appointment duration of Mr. B. In FIG. 7B, Mr. B selects the next timeslot after Mr. A to book the appointment and the scheduling module 108 books the appointment for Mr. B. This prevents anyone else from booking that timeslot, and the scheduling module 108 will not recommend to anyone else and will prevent anyone else from booking that same timeslot of the clinician on the date.

In FIG. 7C, the scheduling module 108 continues to stack and shift the one or more available/future timeslots that can be provided to other patients seeking to schedule an appointment. For example, the scheduling module 108 determined that the greater of Mr. B's T3 or T4 is to be used to define possible starts times of available timeslots for Mr. C. Also, the greater of Mr. C's T3 or T4 is used to determine the predicted duration of the appointment to be scheduled for Mr. C. In the example of FIG. of 7C, Mr. C has a T3 duration that is less that his T4 duration, so T4 is used to determine the predicted appointment duration of Mr. C, and to offer available timeslots offered to Mr. C that can allocate T4 in duration for scheduling. FIG. 7D continues with the scheduling module 108 stacking or shifting the one or more available/future timeslots that can be provided to the patients seeking to schedule an appointment, for example, for Ms. D. As should be understood, FIGS. 7A-D are merely an example and the sequential nature of the patients' selections is not limiting or demonstrative, as each patient's selection may be different and timeslots can be selected in randomly in any order and do not need to be consecutive or sequential. As such, the examples shown FIGS. 7A-D are not limiting and have been simplified merely to provide a clear and easily understood scenario.

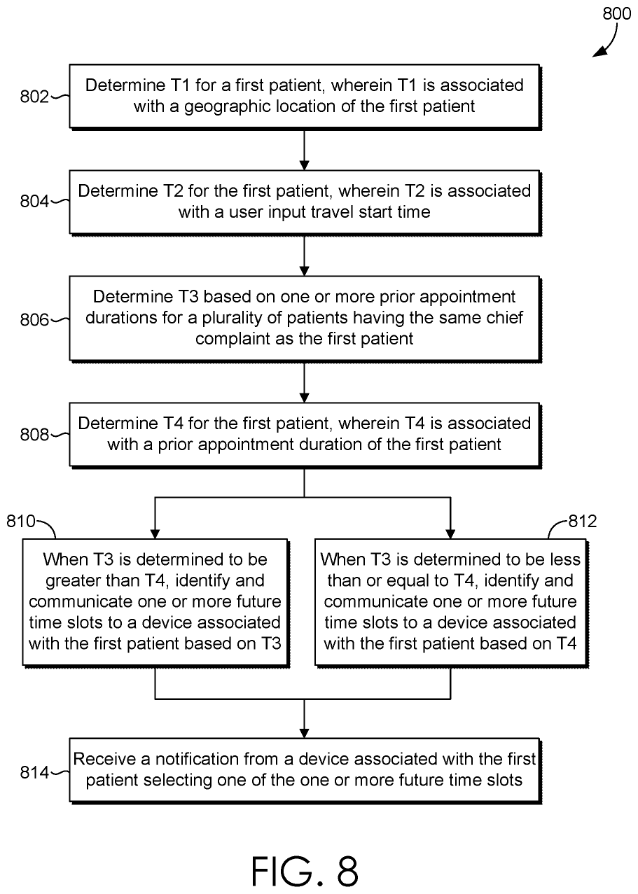

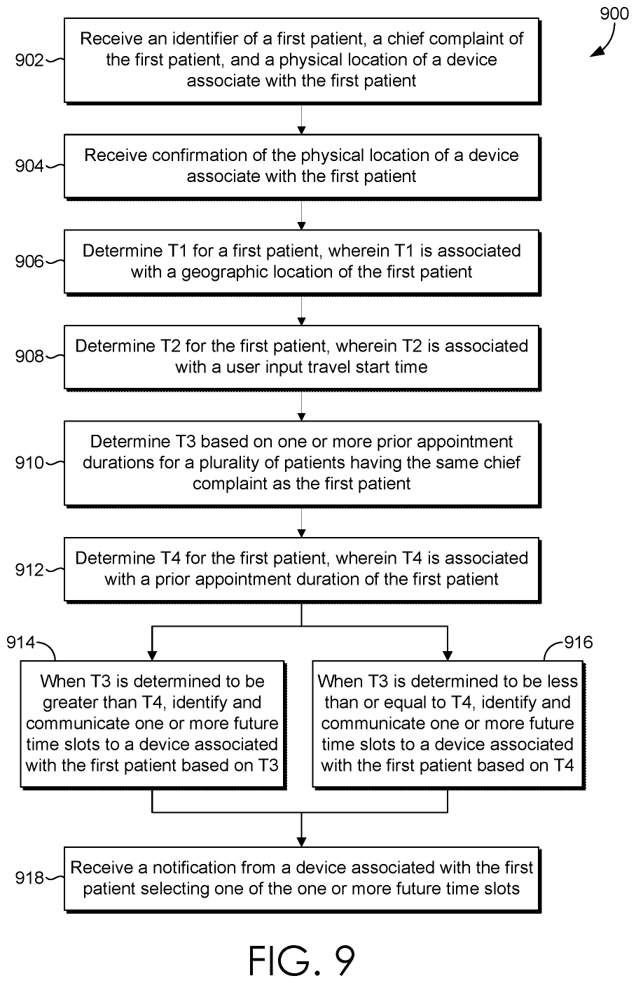

Turning now to FIGS. 8 and 9, methods are discussed that can be performed by way of the system, modules, and interactions described in FIGS. 1-7D. As such, the methods are discussed briefly for brevity, though it will be understood that the previous discussion and details described therein can be applicable to aspect of the methods of FIGS. 8 and 9. Additionally or alternatively, it will be understood that the methods discussed herein can be computer implemented or performed via the execution of computer-readable instructions stored on computer readable media, by one or more processors. For example, one or more non-transitory computer-readable storage medium having computer-readable program code portions embodied can be used to perform and/or implement the methods discussed hereinafter.

FIG. 8 depicts a flow diagram showing a method 800 that leverages near real-time location information of a particular patient and historical appointment data of patient cohorts in order to determine and recommend future appointment start times, as well as to predict an appointment duration for the recommended future appointments. At block 802, T1 is determined for a first patient, wherein T1 is associated with a geographic location of the first patient. T2 is determined for the first patient at block 804, wherein T2 is associated with a user input travel start time. In some aspects, determining T2 comprises calculating an earliest travel start time that the first patient can start from their geographic location. T3 is determined based on one or more prior appointment durations for a plurality of patients having the same chief complaint as the first patient, shown at block 806. In various aspects, determining T3 may include one or more of calculating an average time duration spent by a clinician for the plurality of patients having the same chief complaint, calculating an average time duration follow-up consultation time taken for the plurality of patients having the same chief complaint, or calculating an average time duration per patient to reach the clinical site for the plurality of patients having the same chief complaint. At block 808, T4 is determined for the first patient, wherein T4 is associated with a prior appointment duration of the first patient. In some aspects, T4 is determined by calculating an average time duration for one or more prior appointment of the first patient, wherein the one or more prior appointments for the first patient are utilized for calculating the independent of the chief complaint. A scheduling module can automatically determine T1, T2, T3, and T4, as previously discussed.

When T3 is determined to be greater than T4, shown at block 810, one or more future timeslots are identified based on T3 and are communicated to a device associated with the first patient. When T3 is determined to be less than or equal to T4, shown at block 812, one or more future timeslots are identified based on T4 and are communicated to a device associated with the first patient. Then, at block 814, a notification is received from a device associated with the first patient, wherein the notification includes a selection of one the one or more future timeslots. The appointment is then booked for the one selected future timeslot, for example, by a scheduling module as previously discussed.

FIG. 9 depicts a flow diagram showing a method 900 that leverages near real-time location information of a particular patient and historical appointment data of patient cohorts in order to determine and recommend future appointment start times, as well as to predict an appointment duration for the recommended future appointments. At block 902, an identifier of a first patient, a chief complaint of the first patient, and a geographic location of a device associated with the first patient are received. Further, in aspects, a confirmation of the geographic location of a device associated with the first patient is received, shown at block 904. For example, a patient intake module may receive the first patient identifier, chief complaint, geographic location, and/or confirmation of the geographic location, as previously discussed. At block 906, T1 is determined for the first patient, wherein T1 is associated with a geographic location of the first patient. T2 is determined for the first patient, at block 908, wherein T2 is associated with an input travel start time. The first patient, guardian, or other user booking the appointment on behalf of the first patient may input the travel start time. For example, T2 may be determined by calculating an earliest travel start time that the first patient can start from their geographic location.

T3 is determined, at block 910, based on one or more prior appointment durations for a plurality of patients having the same chief complaint as the first patient. In various aspects, T3 can be determined by calculating an average duration spent by a clinician for the plurality of patients having the same chief complaint, calculating an average duration follow-up consultation time taken for the plurality of patients having the same chief complaint, calculating an average duration per patient to reach the clinical site for the plurality of patients having the same chief complaint, or any combination thereof. T4 is determined for the first patient, a block 912, wherein T4 is associated with a prior appointment duration of the first patient. In some aspects, T4 is determined by calculating an average duration for one or more prior appointment of the first patient, wherein the one or more prior appointments for the first patient are utilized for calculating the independent of the chief complaint. A scheduling module can automatically determine T1, T2, T3, and T4, as previously discussed.

Continuing, when T3 is determined to be greater than T4, shown at block 914, one or more future timeslots are identified and communicated to a device associated with the first patient based on T3. The one or more future wherein the one or more future timeslots begin after T1 or T2, have a duration based on T3, and exclude any previously reserved timeslots, in various aspects. When T3 is determined to be less than or equal to T4, one or more future timeslots are identified and communicated to a device associated with the first patient based on T4. The one or more future wherein the one or more future timeslots begin after T1 or T2, have a duration based on T4, and exclude any previously reserved timeslots, in various aspects. At block 918, a notification is received from a device associated with the first patient, wherein the notification includes a selection of one of the one or more future timeslots. The appointment is then booked for the one selected future timeslot, for example, by a scheduling module as previously discussed.

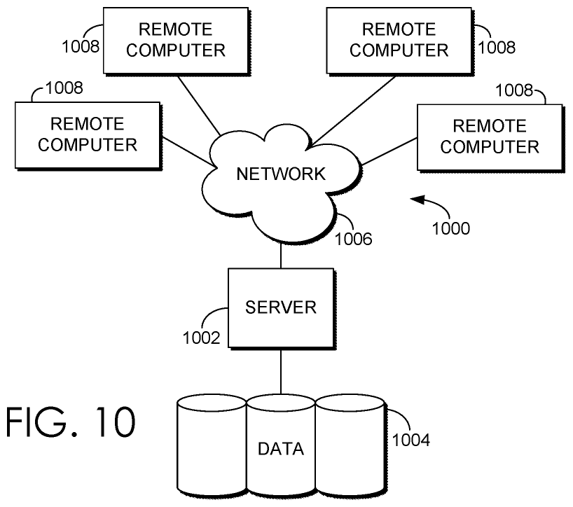

Turning now to with FIG. 10, an exemplary computing environment is depicted, in accordance with one or more aspects of the present invention. It will be understood by those of ordinary skill in the art that the exemplary computing environment 1000 is just one example of a suitable computing environment and is not intended to limit the scope of use or functionality of the present invention. Similarly, the computing environment 1000 should not be interpreted as imputing any dependency and/or any requirements with regard to each component and combination(s) of components illustrated in FIG. 10. It will be appreciated by those having ordinary skill in the art that the connections illustrated in FIG. 10 are also exemplary as other methods, hardware, software, and devices for establishing a communications link between the components, devices, systems, and entities, as shown in FIG. 10, may be utilized in implementation of the present invention. Although the connections are depicted using one or more solid lines, it will be understood by those having ordinary skill in the art that the exemplary connections of FIG. 10 may be hardwired or wireless, and may use intermediary components that have been omitted or not included in FIG. 10 for simplicity's sake. As such, the absence of components from FIG. 10 should be not be interpreted as limiting the present invention to exclude additional components and combination(s) of components. Moreover, though devices and components are represented in FIG. 10 as singular devices and components, it will be appreciated that some aspects may include a plurality of the devices and components such that FIG. 10 should not be considered as limiting the number of a device or component.

Continuing, the computing environment 1000 of FIG. 10 is illustrated as being a distributed environment where components and devices may be remote from one another and may perform separate tasks. The components and devices may communicate with one another and may be linked to each other using a network 1002. The network 1002 may include wireless and/or physical (e.g., hardwired) connections. Exemplary networks include a telecommunications network of a service provider or carrier, Wide Area Network (WAN), a Local Area Network (LAN), a Wireless Local Area Network (WLAN), a cellular telecommunications network, a Wi-Fi network, a short range wireless network, a Wireless Metropolitan Area Network (WMAN), a Bluetooth® capable network, a fiber optic network, or a combination thereof. The network 1002, generally, provides the components and devices access to the Internet and web-based applications.

The computing environment 1000 comprises a computing device 1004, which may be in the form of a server. Although illustrated as one component in FIG. 10, the present invention may utilize a plurality of local servers and/or remote servers in the computing environment 1000. The computing device 1004 may include components such as a processing unit, internal system memory, and a suitable system bus for coupling to various components, including a database or database cluster. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA®) local bus, and Peripheral Component Interconnect (PCI) bus, also known as Mezzanine bus.

The computing device 1004 may include or may have access to computer-readable media. Computer-readable media can be any available media that may be accessed by computing device 1004, and includes volatile and nonvolatile media, as well as removable and non-removable media. By way of example, and not limitation, computer-readable media may include computer storage media and communication media. Computer storage media may include, without limitation, volatile and nonvolatile media, as well as removable and non-removable media, implemented in any method or technology for storage of information, such as computer-readable instructions, data structures, program modules, or other data. In this regard, computer storage media may include, but is not limited to, Random Access Memory (RAM), Read-Only Memory (ROM), Electrically Erasable Programmable Read-Only Memory (EEPROM), flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the computing device 1004. Computer storage media does not comprise signals per se.

Communication media typically embodies computer-readable instructions, data structures, program modules, or other data in a modulated data signal, such as a carrier wave or other transport mechanism, and may include any information delivery media. As used herein, the term “modulated data signal” refers to a signal that has one or more of its attributes set or changed in such a manner as to encode information in the signal. By way of example, and not limitation, communication media includes wired media such as a wired network or direct-wired connection, and wireless media such as acoustic, radio frequency (RF), infrared, and other wireless media. Combinations of any of the above also may be included within the scope of computer-readable media.

In aspects, the computing device 1004 uses logical connections to communicate with one or more remote computers 1006 within the computing environment 1000. In aspects where the network 1002 includes a wireless network, the computing device 1004 may employ a modem to establish communications with the Internet, the computing device 1004 may connect to the Internet using Wi-Fi or wireless access points, or the server may use a wireless network adapter to access the Internet. The computing device 1004 engages in two-way communication with any or all of the components and devices illustrated in FIG. 10, using the network 1002. Accordingly, the computing device 1004 may send data to and receive data from the remote computers 1006 over the network 1002.

Although illustrated as a single device, the remote computers 1006 may include multiple computing devices. In an aspect having a distributed network, the remote computers 1006 may be located at one or more different geographic locations. In an aspect where the remote computers 1006 is a plurality of computing devices, each of the plurality of computing devices may be located across various locations such as buildings in a campus, medical and research facilities at a medical complex, offices or “branches” of a banking/credit entity, or may be mobile devices that are wearable or carried by personnel, or attached to vehicles or trackable items in a warehouse, for example.

In some aspects, the remote computers 1006 is physically located in a medical setting such as, for example, a laboratory, inpatient room, an outpatient room, a hospital, a medical vehicle, a veterinary environment, an ambulatory setting, a medical billing office, a financial or administrative office, hospital administration setting, an in-home medical care environment, and/or medical professionals' offices. By way of example, a medical professional may include physicians; medical specialists such as surgeons, radiologists, cardiologists, and oncologists; emergency medical technicians; physicians' assistants; nurse practitioners; nurses; nurses' aides; pharmacists; dieticians; microbiologists; laboratory experts; genetic counselors; researchers; veterinarians; students; and the like. In other aspects, the remote computers 1006 may be physically located in a non-medical setting, such as a packing and shipping facility or deployed within a fleet of delivery or courier vehicles.

Continuing, the computing environment 1000 includes a data store 1008. Although shown as a single component, the data store 1008 may be implemented using multiple data stores that are communicatively coupled to one another, independent of the geographic or physical location of a memory device. Exemplary data stores may store data in the form of artifacts, server lists, properties associated with servers, environments, properties associated with environments, computer instructions encoded in multiple different computer programming languages, deployment scripts, applications, properties associated with applications, release packages, version information for release packages, build levels associated with applications, identifiers for applications, identifiers for release packages, users, roles associated with users, permissions associated with roles, workflows and steps in the workflows, clients, servers associated with clients, attributes associated with properties, audit information, and/or audit trails for workflows. Exemplary data stores may also store data in the form of electronic records, for example, electronic medical records of patients, transaction records, billing records, task and workflow records, chronological event records, and the like.

Generally, the data store 1008 includes physical memory that is configured to store information encoded in data. For example, the data store 1008 may provide storage for computer-readable instructions, computer-executable instructions, data structures, data arrays, computer programs, applications, and other data that supports the functions and action to be undertaken using the computing environment 1000 and components shown in exemplary FIG. 10.

In a computing environment having distributed components that are communicatively coupled via the network 1002, program modules may be located in local and/or remote computer storage media including, for example only, memory storage devices. Aspects of the present invention may be described in the context of computer-executable instructions, such as program modules, being executed by a computing device. Program modules may include, but are not limited to, routines, programs, objects, components, and data structures that perform particular tasks or implement particular abstract data types. In aspects, the computing device 1004 may access, retrieve, communicate, receive, and update information stored in the data store 1008, including program modules. Accordingly, the computing device 1004 may execute, using a processor, computer instructions stored in the data store 1008 in order to perform aspects described herein.

Although internal components of the devices in FIG. 10, such as the computing device 1004, are not illustrated, those of ordinary skill in the art will appreciate that internal components and their interconnection are present in the devices of FIG. 10. Accordingly, additional details concerning the internal construction device are not further disclosed herein.

The present invention has been described in relation to particular aspects, which are intended in all respects to be illustrative rather than restrictive. Further, the present invention is not limited to these aspects, but variations and modifications may be made without departing from the scope of the present invention.

What is claimed is:

1. A computerized method for leveraging near real-time location information and historical appointment data of patient cohorts to calculate future appointment start times and predicted durations, the computerized method comprising:
determining T1 for a first patient, wherein T1 is associated with a geographic location of the first patient;
determining T2 for the first patient, wherein T2 is associated with a user input travel start time;
determining T3 based on one or more prior appointment durations for a plurality of patients having the same chief complaint as the first patient;
determining T4 for the first patient, wherein T4 is associated with a prior appointment duration of the first patient;
when T3 is determined to be greater than T4, identifying and communicating one or more future timeslots to a device associated with the first patient based on T3;
when T3 is determined to be less than or equal to T4, identifying and communicating one or more future timeslots to the device associated with the first patient based on T4; and
receiving a notification from the device associated with the first patient, wherein the notification includes a selection of one of the one or more future timeslots.

2. The method of claim 1, wherein determining T3 comprises calculating an average time duration spent by a clinician for the plurality of patients having the same chief complaint.

3. The method of claim 1, wherein determining T3 comprises calculating an average time duration follow-up consultation time taken for the plurality of patients having the same chief complaint.

4. The method of claim 1, wherein determining T3 comprises calculating an average time duration per patient to reach a clinical site for the plurality of patients having the same chief complaint.

5. The method of claim 1, wherein determining T4 comprises calculating an average time duration for one or more prior appointments of the first patient, wherein the one or more prior appointments for the first patient are utilized for calculating T4 independent of the chief complaint.

6. The method of claim 1, wherein determining T2 comprises calculating an earliest travel start time that the first patient can start from the geographic location.

7. One more non-transitory computer-readable media having computer-executable instructions embodied thereon that, when executed, the media comprising:
receive an identifier of a first patient, a chief complaint of the first patient, and a geographic location of a device associated with the first patient;
receive confirmation of the geographic location of the device associated with the first patient;
determine T1 for the first patient, wherein T1 is associated with the geographic location of the first patient;
determine T2 for the first patient, wherein T2 is associated with an input travel start time;
determine T3 based on one or more prior appointment durations for a plurality of patients having the same chief complaint as the first patient;
determine T4 for the first patient, wherein T4 is associated with a prior appointment duration of the first patient;
when T3 is determined to be greater than T4, identify and communicate one or more future timeslots to the device associated with the first patient based on T3;
when T3 is determined to be less than or equal to T4, identify and communicate one or more future timeslots to the device associated with the first patient based on T4; and
receive a notification from the device associated with the first patient, wherein the notification includes a selection of one of the one or more future timeslots.

8. The media of claim 7, wherein T3 is determined by calculating an average time duration spent by a clinician for the plurality of patients having the same chief complaint.

9. The media of claim 7, wherein determining T3 comprises calculating an average time duration follow-up consultation time taken for the plurality of patients having the same chief complaint.

10. The media of claim 7, wherein T3 is determined by calculating an average time duration per patient to reach a clinical site for the plurality of patients having the same chief complaint.

11. The media of claim 7, wherein T4 is determined by calculating an average time duration for one or more prior appointments of the first patient, wherein the one or more prior appointments for the first patient are utilized for calculating the independent of the chief complaint.

12. The media of claim 7, wherein T2 is determined by calculating an earliest travel start time that the first patient can start from the geographic location.

13. The media of claim 7, wherein when T3 is determined to be less than or equal to T4, the one or more future timeslots are identified as having a start time based on T1+T2 and a duration based on T4.

14. The media of claim 7, wherein when T3 is determined to be greater than T4, the one or more future timeslots are identified as having a start time based on T1+T2 and a duration based on T3.

15. A system for leveraging near real-time location information and historical appointment data of patient cohorts to calculate future appointment start times and predicted durations, the system comprising:
a processor;
a memory;
a patient intake module; and
a scheduling module;
wherein the patient intake module:
receives an identifier of a first patient, a chief complaint of the first patient, and a geographic location of a device associated with the first patient;
receives confirmation of the geographic location of the device associated with the first patient;
wherein the scheduling module:
determines T1 for the first patient, wherein T1 is associated with the geographic location of the device associated with the first patient;
determines T2 for the first patient, wherein T2 is associated with an input travel start time;
determines T3 based on one or more prior appointment durations for a plurality of patients having the same chief complaint as the first patient;
determines T4 for the first patient, wherein T4 is associated with a prior appointment duration of the first patient;
when T3 is determined to be greater than T4, identifies and communicates one or more future timeslots to the device associated with the first patient based on T3;
when T3 is determined to be less than or equal to T4, identifies and communicates one or more future timeslots to the device associated with the first patient based on T4; and
receives a notification from the device associated with the first patient, wherein the notification includes a selection of one of the one or more future timeslots.

16. The system of claim 15, wherein the scheduling module determines T3 by calculating an average time duration spent by a clinician for the plurality of patients having the same chief complaint.

17. The system of claim 15, wherein the scheduling module determines T3 by calculating an average time duration follow-up consultation time taken for the plurality of patients having the same chief complaint.

18. The system of claim 15, wherein the scheduling module determines T3 by calculating an average time duration per patient to reach a clinical site for the plurality of patients having the same chief complaint.

19. The system of claim 15, wherein the scheduling module determines T4 by calculating an average time duration for one or more prior appointments of the first patient, wherein the one or more prior appointments for the first patient are utilized for calculating the independent of the chief complaint.

20. The system of claim 15, wherein the scheduling module determines T2 by calculating an earliest travel start time that the first patient can start from the geographic location.
